(19) United States
(12) Patent Application Publication
(10) Pub. No.: US 2026/0091203 A1
(43) Pub. Date: Apr. 2, 2026

(54) MEDICAL DEVICE AND METHOD FOR PRODUCING THE SAME

(71) Applicant: ASAHI INTECC CO., LTD., Seto-shi (JP)

(72) Inventors: Moritaka OGIDO, Aichi (JP); Sayako WATANABE, Aichi (JP)

(73) Assignee: ASAHI INTECC CO., LTD., Seto-shi (JP)

(21) Appl. No.: 19/411,390

(22) Filed: Dec. 8, 2025

Related U.S. Application Data
(63) Continuation of application No. PCT/JP2024/018576, filed on May 21, 2024.

(30) Foreign Application Priority Data
Jun. 21, 2023 (JP) 2023-101745

(51) Int. Cl. A61M 25/06; A61M 25/00

(52) U.S. Cl. CPC A61M 25/0662; A61M 25/0012; A61M 25/0015; A61M 25/0045; A61M 25/005; A61M 2205/586; A61M 2207/00

(57) ABSTRACT

A medical device includes a reinforcing member braided into a tubular shape using a wire and an inner layer disposed so as to be in contact with the radially inner side of the reinforcing member. A lumen is formed on the radially inner side of the inner layer. The inner layer includes a first region formed of a first material, and a second region, provided in a portion other than the first region, having an inner peripheral surface with a sliding resistance greater than that of the inner peripheral surface of the first region, and formed of a second material different from the first material.

### CROSS-REFERENCE TO RELATED APPLICATIONS

This application is a continuation application of International Application No. PCT/JP2024/018576, filed May 21, 2024, which claims priority to Japanese Patent Application No. 2023-101745, filed Jun. 21, 2023. The contents of these applications are incorporated herein by reference in their entirety.

### TECHNICAL FIELD

The present disclosure relates to a medical device and a method for producing the same.

### BACKGROUND

When guiding a medical instrument (combination-use device) such as a guidewire or a catheter from outside the body into a body cavity such as a blood vessel, for example, a medical device such as a guiding catheter is used.

As such a medical device needs to quickly guide the combination-use device into the body cavity, a technique has been proposed in which an inner layer with low friction (sliding resistance) is provided on the inner peripheral surface that comes into contact with the combination-use device, so as to reduce the sliding resistance with the combination-use device inserted into the lumen (see, e.g., JP H8-168521 A).

According to the above-described technique, for example, the entire inner layer is formed using a material such as polytetrafluoroethylene (PTFE) or a tetrafluoroethylene-perfluoroalkyl vinyl ether copolymer (PFA), thereby reducing the sliding resistance with the combination-use device on the inner peripheral surface.

### SUMMARY

However, as described above, while reducing the sliding resistance makes it possible to quickly guide the combination-use device into the body cavity, it also tends to make the combination-use device more prone to positional displacement within the medical device, making it difficult to, for example, rotate the guidewire at the desired location or position the balloon of a balloon catheter at the desired location.

The present disclosure has been made in light of the above circumstances, and provides a medical device that can temporarily grip a combination-use device inserted into a lumen, and a method for producing the same.

A medical device according to one aspect of the present disclosure includes a reinforcing member braided in a tubular shape using a wire, and an inner layer disposed so as to be in contact with the radial inner side of the reinforcing member. A lumen is formed on the radial inner side of the inner layer, and the inner layer includes a first region formed of a first material, and a second region, provided in a portion other than the first region, having an inner peripheral surface with a sliding resistance greater than a sliding resistance of the inner peripheral surface of the first region, and formed of a second material different from the first material.

Note that, in the present specification, the term “distal side” refers to a direction along the longitudinal axis of the medical device and is a direction in which the medical device is inserted deeper (into distal) within the body. Further, the term “proximal side” refers to a direction along the longitudinal axis of the medical device opposite to the distal side. Further, the terms “distal end” and “distal end portion” refer to a portion on the distal side of any member or portion, while “proximal end” and “proximal end portion” refer to a portion on the proximal side of any member or portion. These portions do not necessarily include the edge. Unless otherwise specified, the term “longitudinal direction” refers to a direction along the central axis (tube axis) of the medical device. The term “radial direction” refers to the radial direction orthogonal to the longitudinal direction of the medical device.

### BRIEF DESCRIPTION OF DRAWINGS

FIG. 1 is a schematic longitudinal cross-sectional view illustrating the entirety of an embodiment of a medical device.

FIG. 2 is a schematic longitudinal cross-sectional view illustrating an enlarged portion of FIG. 1.

FIG. 3A is a schematic cross-sectional view taken along a line IIIA-IIIA in FIG. 2.

FIG. 3B is a schematic cross-sectional view taken along a line IIIB-IIIB in FIG. 2.

FIG. 4 is a schematic longitudinal cross-sectional view illustrating an enlarged portion of another embodiment of the medical device.

FIG. 5A is a schematic cross-sectional view taken along a line VA-VA in FIG. 4.

FIG. 5B is a schematic cross-sectional view taken along a line VB-VB in FIG. 4.

FIG. 6A is an explanatory diagram of an embodiment of a method for producing a medical device.

FIG. 6B is an explanatory diagram of an embodiment of the method for producing the medical device.

FIG. 6C is an explanatory diagram of an embodiment of the method for producing the medical device.

FIG. 6D is an explanatory diagram of an embodiment of the method for producing the medical device.

FIG. 6E is an explanatory diagram of an embodiment of the method for producing the medical device.

### DETAILED DESCRIPTION

Hereinafter, embodiments are described with reference to the drawings. However, the present disclosure is not limited to the embodiments described in the drawings. In addition, the dimension of each portion illustrated in the drawings is provided for ease of understanding of the embodiments and does not necessarily correspond to the actual dimension.

### <Medical Device>

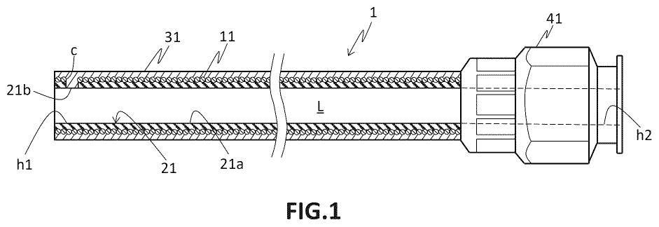

FIG. 1 to FIG. 3 are schematic cross-sectional views illustrating one embodiment of a medical device 1. The medical device 1 is, for example, a guiding catheter, but may be used with other tubular medical devices such as microcatheters or guide sheaths, as will be apparent from this disclosure. As illustrated in FIG. 1 to FIG. 3, the medical device 1 is generally configured by a reinforcing member 11, an inner layer 21, an outer layer 31, and a gripping member 41.

The reinforcing member 11 is a member braided in a tubular shape using a wire. Specifically, the reinforcing member 11 can be formed, for example, by braiding a wire w into a mesh pattern while shaping the whole into a tubular shape or by winding the wire w helically around the longitudinal axis of the medical device 1 while shaping it into a tubular shape. By providing the reinforcing member 11, the medical device 1 can, for example, hold the inner layer 21 described below and increase the overall strength of the medical device 1.

The wire w may be a single wire, a stranded wire, or a combination of single and stranded wires. Here, the term “single wire” refers to one individual wire, while the term “stranded wire” refers to a bundle of wires formed in advance by twisting together multiple individual wires.

A material of the wire w may be a metal material, a resin material, a composite material thereof, or the like. Examples of the metal material include, but are not limited to: stainless steel such as SUS304; a superelastic alloy such as a Ni—Ti alloy; and a radiopaque metal such as platinum or tungsten. Examples of the resin material include, but are not limited to, nylon and polyethylene.

The reinforcing member 11 may be provided at a location on the radially outer side of a second region 21b (described below) and may have a predetermined passage c communicating in the radial direction. By providing the passage c, for example, when forming the second region 21b described below, a second material m2 can be easily loaded (placed) from the radial outer side through the passage c.

The inner layer 21 is a member disposed in contact with the radially inner side of the reinforcing member 11. The inner layer 21 includes a first region 21a and the second region 21b. A lumen h1, which penetrates from the distal end to the proximal end, is formed on the radially inner side of the inner layer 21.

The first region 21a is a portion formed of a first material m1. Specifically, the first region 21a may be provided, for example, in one or more separate portions of the inner layer 21. The first region 21a may be formed in an annular shape along the circumferential direction of the inner layer 21, or it may be formed only in a part of the circumferential direction.

The second region 21b is provided at a portion other than the first region 21a, has a sliding resistance on its inner peripheral surface greater than that of the inner peripheral surface of the first region 21a, i.e., the second region 21b has a higher coefficient of friction than the first region 21a, and is formed of the second material m2 different from the first material m1. Specifically, the second region 21b may be provided, for example, in one or more separate portions of the inner layer 21. The second region 21b may be formed in an annular shape along the circumferential direction of the inner layer 21, or it may be formed only in a part of the circumferential direction. This greater sliding resistance allows the second region 21b to function as a temporary gripping zone, providing enhanced positional control of the combination-use device for an operator.

The arrangement of the first region 21a and the second region 21b in the inner layer 21 is not particularly limited. For example, the second region 21b may be provided only at an end portion in the longitudinal direction, or it may be provided at an intermediate position. Such arrangements can be appropriately determined according to the position within the medical device at which the inserted combination-use device is to be gripped.

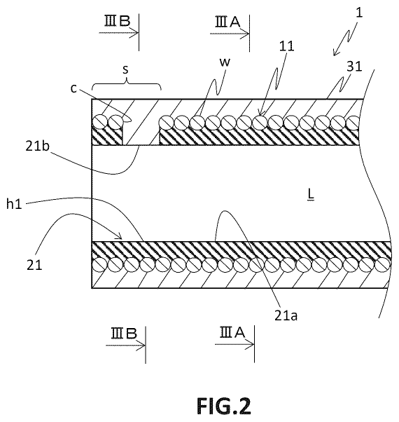

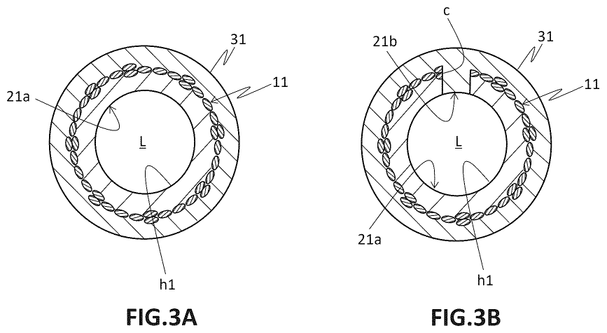

In the present embodiment, the second region 21b is provided only in a part of the circumferential direction at a distal end portion s of the inner layer 21 in the longitudinal direction (see FIG. 2 and FIG. 3B). On the other hand, the first region 21a is provided in the portions of the inner layer 21 other than the second region 21b (see FIG. 2 and FIG. 3A).

If the second region 21b is provided at the distal end portion of the medical device, for example, after quickly inserting the combination-use device to the distal end portion of the medical device, the combination-use device can be gripped at the distal end portion, allowing the position of the combination-use device to be finely adjusted with high accuracy without excessive movement. On the other hand, if the second region 21b is provided at the proximal end portion of the medical device, the combination-use device can be inserted into the medical device slowly and securely. Further, if the second region 21b is provided in an intermediate position of the medical device, the combination-use device, such as a balloon catheter, in which the frictional resistance of the outer peripheral surface varies in the longitudinal direction, can be temporarily held at a specific position within the lumen.

The outer layer 31 is a member formed of the second material m2 and disposed so as to cover the radially outer side of the reinforcing member 11. The outer layer 31 is connected to the second region 21b via the passage c. In other words, the second region 21b of the inner layer 21, formed of the second material m2, and the outer layer 31, also formed of the second material m2, are integrally configured, e.g., monolithically formed, via a portion within the passage c that is formed of the second material m2. Accordingly, each of the inner layer 21 and the outer layer 31 can be securely fixed to the reinforcing member 11 through mutual bonding via the portion within the passage c, in combination with the anchoring effect into the interstices of the wire w that constitutes the reinforcing member 11. Further, by forming the outer peripheral surface of the outer layer 31 smoothly, frictional resistance with a blood vessel or the like can be reduced, enabling the medical device to be advanced and withdrawn smoothly.

Examples of the first material m1 constituting the first region 21a include, but are not limited to, polytetrafluoroethylene (PTFE), fluorinated ethylene propylene (FEP), and a tetrafluoroethylene-perfluoroalkyl vinyl ether copolymer (PFA).

Examples of the second material m2 constituting the second region 21b include, but are not limited to, polyethylene, nylon, polyether block amide, polyethylene terephthalate (PET), and polyether ether ketone (PEEK).

As an example of such a combination, the first material m1 is polytetrafluoroethylene (PTFE), and the second material m2 is polyethylene or nylon. This allows the combination-use device to be advanced and withdrawn easily and quickly when the combination-use device passes through the first region 21a, while providing greater sliding resistance when the combination-use device passes through the second region 21b than through the first region 21a, thereby enhancing the gripping force on the combination-use device.

The gripping member 41 is a member used by the operator to hold and operate the medical device 1. The gripping member 41 can be provided such that it is connected to the proximal end portion of the reinforcing member 11. While the shape of the gripping member 41 is not particularly limited, for example, it may be configured to have a lumen h2 that communicates with the lumen h1 and extends through from the distal end to the proximal end. In such a case, a lumen L is formed by the lumen h1 and the lumen h2. During an operation, a combination-use device such as a guidewire or a catheter is inserted through the lumen L.

Note that a method for producing the medical device 1 is described in detail in the section “Method for producing medical device”.

As described above, since the medical device 1 has the above configuration, the inner peripheral surface of the second region 21b has higher sliding resistance than that of the first region 21a, allowing the combination-use device inserted into the lumen h1 to be temporarily gripped by the second region 21b. As a result, by disposing the second region 21b at a desired location of the inner layer 21, it is possible, for example, to rapidly insert the combination-use device while performing accurate positioning, or to temporarily retain the combination-use device at an appropriate location within the lumen h1.

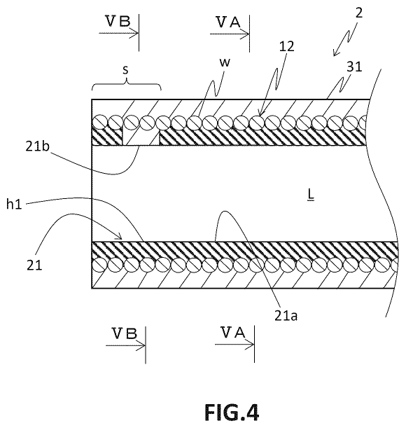

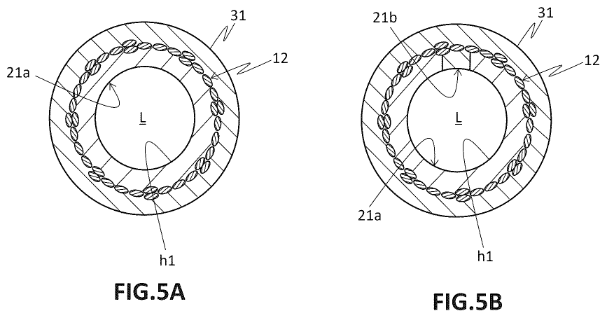

Note that in the embodiment described above, the reinforcing member 11 having the predetermined passage c has been described. However, the reinforcing member does not necessarily need to have the predetermined passage. Even when the second material m2 is loaded (placed) from the radially outer side, as a medical device 2 having a reinforcing member 12 without the passage c illustrated in FIG. 4 and FIG. 5B, the second material m2 can be loaded (placed) through gaps between the adjacent braided wires w of the reinforcing member 11.

### <Method for Producing Medical Device>

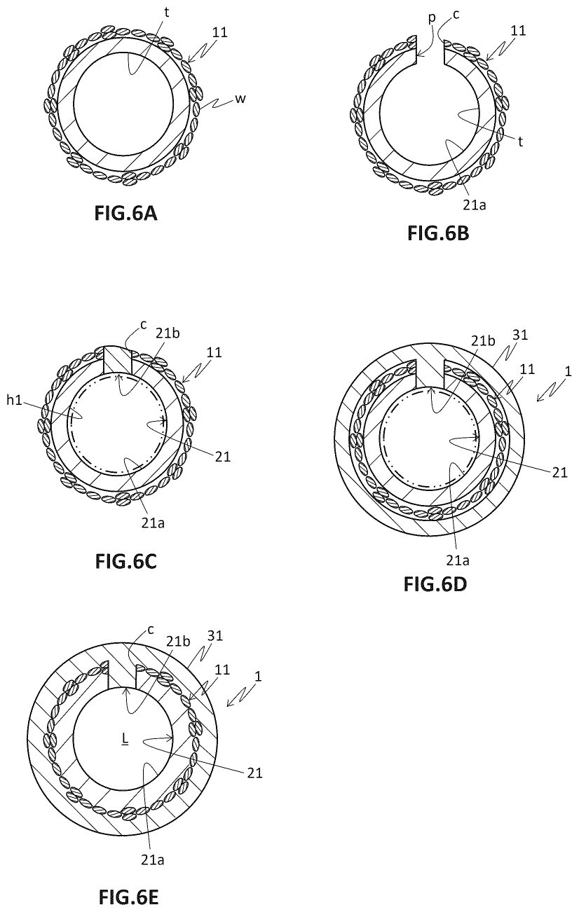

A method of the present disclosure is carried out in the order of a first step, a second step, and a third step. Below, with reference to FIG. 6A to FIG. 6E, the method for producing a medical device is described in detail. Here, the description is given by way of examples using the medical device 1 (see FIG. 1, FIG. 2, FIG. 3A, and FIG. 3B) provided with the passage c and the outer layer 31. FIG. 6A to FIG. 6E each illustrates a schematic cross-sectional view of the portions corresponding to FIG. 3B.

### [First Step]

The first step is a step in which the tubular reinforcing member 11 braided using the wire w is used, and a tube-shaped member t (hereinafter also simply referred to as “tube t”) formed of the first material m1 is disposed on the radially inner side of the reinforcing member 11.

As the reinforcing member 11, for example, one formed by braiding the wire w into a mesh pattern and shaping the whole into a tubular shape, or one formed by winding the wire w helically around the longitudinal axis of the medical device to form a tubular shape, can be used. In the present embodiment, the tubular reinforcing member 11 in which the wire w is braided into a mesh pattern is exemplified.

In the first step, as illustrated in FIG. 6A, specifically, the tube t formed of the first material m1 is used, and is inserted into the inner space of the above-described tubular reinforcing member 11 so that the inner peripheral surface of the reinforcing member 11 is in contact with the outer peripheral surface of the tube t. In the longitudinal direction, end portions of the reinforcing member 11 and the tube t may be aligned. However, it is sufficient that, at least in the portion where a through-hole p described below is formed, the reinforcing member 11 and the tube t are disposed in contact with each other.

### [Second Step]

The second step, following the first step, is a step in which a through-hole p penetrating the tube t is formed to define the first region 21a.

A method for forming the through-hole p is not particularly limited. Examples of the method for forming the through-hole p include, but are not limited to, a method of mechanically boring the through-hole p using a drill or a cutter of predetermined shape, and a method of thermally boring the through-hole p by applying laser light (method of causing evaporation by heating).

In the present embodiment, as shown in FIG. 6B, a laser irradiation device is used to apply laser light from the radial outer side only onto a predetermined portion of the reinforcing member 11, thereby forming the through-hole p only in a part of the circumferential direction at the distal end portion s of the inner layer 21 in the longitudinal direction. This defines the first region 21a (a portion of the tube t other than the portion where the through-hole p is formed). Further, in the present embodiment, the through-hole p in the tube t and the passage c in the reinforcing member 11 are formed simultaneously by laser light irradiation. Note that the type and intensity of the laser light to be applied, as well as the application range, can be appropriately determined to form the through-hole p and the like.

### [Third Step]

The third step, following the second step, is a step in which a second material m2, different from the first material m1, is used to form the second region 21b while filling the through-hole p with the second material m2.

In the third step, as shown in FIG. 6C, specifically, for example, the second material m2 in a heated and molten state is used, and the molten second material m2 is filled into the through-hole p to define the second region 21b. At this time, the filling may be performed after a mandrel (shown by a two-dot chain line in FIG. 6C) corresponding to the diameter of the lumen h1 to be formed (the inner diameter of the tube t) is inserted in advance into the inner space of the tube t. This enables the inner peripheral surface of the inner layer 21 to be formed flush (smooth inner peripheral surface without a step at a boundary between the first region 21a and the second region 21b in the longitudinal direction), and can prevent the excessive overflow of the second material m2, thereby ensuring that the second region 21b is formed only in the predetermined portion.

The third step of the present embodiment also includes a step of forming the outer layer 31 by using the second material m2, different from the first material m1, to cover the radially outer side (outer peripheral surface) of the reinforcing member 11. This step may be performed after filling the through-hole p with the second material m2, or, as shown in FIG. 6D, may be performed simultaneously with the filling of the through-hole p with the second material m2. When the formation of the outer layer 31 and the filling of the through-holes p are performed simultaneously, for example, after placing a tube formed of the second material m2 over the reinforcing member 11, a portion of the tube corresponding to the through-holes p is heated and melted, thereby allowing the second material m2 to be filled into the through-hole p through the passage c and the gaps of the adjacent braided wires w.

Further, after performing the above filling of the second material m2, the first material m1 and the second material m2 may be heated together in a heating furnace so as to integrate the two materials and bring them into close contact with the reinforcing member 11 (see FIG. 6E).

Further, during or after the above steps, the reinforcing member 11, the inner layer 21, and the like (also the outer layer 31, if provided) may be cut to a predetermined length, and the gripping member 41 may be attached, as needed. Methods for performing the cutting and attachment are not particularly limited, and may be carried out, for example, using known techniques.

As described above, the method for producing a medical device of the present disclosure, having the above configuration, makes it possible to easily and reliably form the inner layer 21 that includes the first region 21a formed of the first material m1 and the second region 21b formed of the second material m2.

Further, the method for producing a medical device of the present disclosure, having the above configuration, can be suitably used for producing the medical device of the present disclosure.

It is to be understood that the present disclosure is not limited to the configuration of the above-described embodiment, but is defined by the claims, and is intended to include all modifications within the equivalent meaning and scope of the claims. Part of the configuration of the above-described embodiment may be omitted or replaced with another configuration, and another configuration may also be added to the configuration of the above-described embodiment.

For example, in the embodiments of the medical device and its production method described above, an example was given in which the second region 21b is formed only in a part of the circumferential direction of the inner layer 21. However, the second region may, for example, be formed as an annular portion along the circumferential direction, or may be formed as two or more separate arc-shaped portions along the circumferential direction.

In the embodiments of the medical device and its production method described above, an example was given of the medical device in which the through-hole is formed in only one location. However, the through-hole may be formed, as appropriate (e.g., in two or more locations), in correspondence with the locations of the second regions.

Further, in the embodiment of the medical device described above, the medical device 1 provided with the passage c and the outer layer 31 was described. However, the medical device may be the medical device 2 without the passage c (see FIG. 4, FIG. 5A, and FIG. 5B) or without the outer layer 31 (see FIG. 6C). The medical device may also be a microcatheter, a guide sheath, or the like.

Further, in the embodiment of the method for producing the medical device 1 described above, an example including the first to third steps was described. However, the medical device may also be produced by another method. As another method, for example, a tube formed in advance by connecting the first region 21a and the second region 21b may be used. This tube is inserted into the inner space of the tubular reinforcing member 11 so that the inner peripheral surface of the reinforcing member 11 comes into contact with the outer peripheral surface of the tube. The tube may then be fixed to the reinforcing member 11 while being softened in a heating furnace. In this manner, the inner layer 21 disposed so as to be in contact with the radially inner side of the reinforcing member 11 can be obtained.

The present disclosure includes the following:

(1) A medical device including:
a reinforcing member braided into a tubular shape using a wire; and
an inner layer disposed so as to be in contact with the radially inner side of the reinforcing member, in which:
a lumen is formed on the radially inner side of the inner layer; and
the inner layer includes:
   a first region formed of a first material; and
   a second region, provided in a portion other than the first region, having an inner peripheral surface with a sliding resistance greater than a sliding resistance of the inner peripheral surface of the first region, and formed of a second material different from the first material.

(2) The medical device according to the (1), in which the reinforcing member includes:
a predetermined passage communicating in the radial direction; and
further comprising an outer layer, formed of the second material, connected to the second region via the passage, and disposed so as to cover the radially outer side of the reinforcing member.

(3) The medical device according to the (1) or (2), in which the second region is provided only at an end portion in the longitudinal direction.

(4) A method for producing the medical device according to any one of the (1) to (3), including:
a first step of using a tubular reinforcing member braided using a wire and disposing a tubular member formed of a first material on the radially inner side of the reinforcing member;
a second step, after the first step, of defining the first region while forming a through-hole penetrating the tubular member, and
a third step, after the second step, of using a second material different from the first material to form the second region while filling the through-hole with the second material.

What is claimed is:

1. A medical device comprising:
a reinforcing member braided into a tubular shape using a wire; and
an inner layer disposed so as to be in contact with a radially inner side of the reinforcing member, wherein:
a lumen is formed on the radially inner side of the inner layer; and
the inner layer includes:
a first region formed of a first material; and
a second region, distinct from the first region, having an inner peripheral surface with a sliding resistance greater than a sliding resistance of the inner peripheral surface of the first region, and formed of a second material different from the first material.

2. The medical device according to claim 1, wherein:
the reinforcing member includes a predetermined passage communicating in a radial direction; and
further comprising
an outer layer, formed of the second material, connected to the second region via the passage, and disposed so as to cover a radially outer side of the reinforcing member.

3. The medical device according to claim 2, wherein each of the inner layer and the outer layer are fixed to the reinforcing member via the predetermined passage.

4. The medical device according to claim 1, wherein the second region is provided only at an end portion in a longitudinal direction.

5. The medical device according to claim 4, wherein the end portion is a distal end portion of the inner layer.

6. The medical device according to claim 3, wherein the end portion is a proximal end portion of the inner layer.

7. The medical device according to claim 1, wherein the second region is annular and extends around an entire circumference of the lumen.

8. The medical device according to claim 1, wherein the second region is arc-shaped and extends around only a portion of a circumference of the lumen.

9. The medical device according to claim 1, wherein the reinforcing member includes a wire helically wound around a longitudinal axis of the medical device.

10. A method for producing a medical device, comprising:
providing a tubular reinforcing member braided using a wire;
disposing a tubular member formed of a first material on a radially inner side of the reinforcing member;
forming a through-hole penetrating the tubular member, wherein a portion of the tubular member not penetrated by the through-hole defines a first region; and
filling the through-hole with the second material different from the first material to form a second region.

11. The method according to claim 10, further comprising, during filling the through-hole, inserting a mandrel within the tubular member to maintain an inner diameter of the lumen.

12. The method according to claim 10, further comprising, after filling the through-hole, heating the first material and the second material to integrate the inner layer with the reinforcing member.
